(19) United States
(12) Patent Application Publication
(10) Pub. No.: US 2022/0143300 A1
(43) Pub. Date: May 12, 2022

(54) FLUSHING VAGINAL STENT DEVICES AND RELATED SYSTEMS AND METHODS

(71) Applicant: REGENTS OF THE UNIVERSITY OF MINNESOTA, MINNEAPOLIS, MN (US)

(72) Inventors: Lyndsey Calvin, Saint Paul, MN (US); Yasheen Brijlal, Minneapolis, MN (US); Nicholas Kim, Minneapolis, MN (US); Amy Lynn Hoelscher, Maplewood, MN (US); Molly KAISSAR, Minneapolis, MN (US)

(21) Appl. No.: 17/593,214

(22) PCT Filed: Mar. 13, 2020
(86) PCT No.: PCT/US2020/022704

§ 371 Date: Sep. 13, 2021

Related U.S. Application Data
(60) Provisional application No. 62/817,819, filed on Mar. 13, 2019.

(51) Int. Cl. A61M 3/02

(52) U.S. Cl. CPC A61M 3/0279; A61M 2210/1475

(57) ABSTRACT

Flushing vaginal stent devices and related systems and methods for supporting and caring for vagina after genital surgery or radiation therapy are disclosed. In an embodiment, a flushing stent device includes an outer component that includes a proximal end aperture fluidly coupled between a douching tube portion internal to the outer component and a plurality of channels formed in an exterior surface of the flushing vaginal stent device, a middle component including a fenestrated stent structure, an inner component including an expansion and retraction mechanism, and a suturing tab portion coupled with at least the inner component.

### RELATED APPLICATION

The present application claims the benefit of U.S. Provisional Application No. 62/817,819 filed Mar. 13, 2019, which is hereby incorporated herein in its entirety by reference.

### TECHNICAL FIELD

Embodiments relate generally to stents and more particularly to flushing vaginal stents for supporting, and preventing stenosis and infection of, a vagina after genital surgery, radiation, or another procedure.

### BACKGROUND

Roughly 3% of people identify as transgender or non-binary. This translates to about 9.9 million people in the United States, and about 234 million worldwide. Of this population, 44.4 million people identify as transfeminine and seek medical transition in the form of vaginoplasty. Currently the medical community is able to provide 20,000 gender affirming vaginoplasties per year. This number is growing between 20-40% each year. This population has historically been marginalized and their healthcare needs ignored. The population of women who need vaginoplasty is clearly large and growing rapidly.

Indeed, there are multiple vulnerable populations that are at risk for vaginal stenosis and infection after genital surgery or radiation. These populations include, but are not limited to, transwomen who have undergone vaginoplasty, patients with pelvic organ cancer requiring radiation therapy or genital surgery, and patients with pediatric genital disorders requiring surgical intervention. Unfortunately, the procedures often result in severe complications such as vaginal stenosis or narrowing of the newly constructed vaginal canal. Other complications include surgical site infection, vaginal prolapse, local abscess, and necrosis.

To combat stenosis and infection (the two most common complications costing over $6.6 billion dollars annually around the world), various medical treatments and inventions have been used in both the acute (short-term) prevention and chronic (long-term) prevention. The most common acute prevention is vaginal packing, which involves packing the neovagina or post-operative vagina with lubricated and antiseptic gauze for the first several (e.g., five) days after the surgery, and then removing it. As the newly constructed vagina is delicate and there is excess scar tissue that may heal into the gauze, removing the packed gauze can be very painful and result in focal necrosis from uneven pressure, infection, and stenosis of the vaginal canal and introitus (external opening of vagina) opening.

After gauze removal, chronic prevention (from, e.g., day six through day ninety, or for some other time period deemed suitable by a physician) includes dilation (expansion of the vaginal canal) and douching (flushing the vaginal canal with an antiseptic fluid such as povidone-iodine) with a dilator (rigid phallic-shaped cylinder), typically three times daily for 30 minutes. The next period (e.g., six to nine months) involves dilating twice a day for the same period. Then, for the rest of her life, the woman must dilate once a day for the same period. This daily dilation is followed by douching, or washing, of the vaginal canal. This is a time-intensive and painful process and can impinge on a woman's ability to work effectively for eight hours or live comfortably. This lengthy and painful process makes dilation and douching difficult to comply with, but noncompliance can result in the same severe consequences as after the surgery or during acute prevention.

Some doctors provide ad hoc solutions to these problems, such as packing a condom with gauze and inserting that into the vagina for the first five days, but these still result in uneven pressure distribution and complications.

### SUMMARY

Therefore, there is a need for improved devices, systems and methods for a flushing stent to be used in supporting and caring for a newly constructed vagina.

Embodiments relate to stents and more particularly to flushing vaginal stents with flushing structures incorporated within the stent for supporting and preventing stenosis and infection of a vagina after genital surgery or radiation therapy. In one embodiment, the stent comprises three components: an inner component that is rigid and removable after the first several postoperative days and comprises an external suturing tab portion for suturing the device to the perineum and vulva after surgery; a middle component that is semi-rigid with a fenestrated or lattice-like structure that remains inserted for a longer period of time (e.g., up to about thirty days), and an outer component that is smooth, non-adhering and flexible and also remains inserted for the longer period of time (e.g., up to about thirty days).

The outer component provides protection from tissue adhesion to the stent and includes a plurality of channels at the proximal (internal) end to encourage even fluid distribution down to the distal (vaginal opening) end during douching. The middle component provides longer-term stent support, with consistent supportive pressure throughout the canal while allowing for minor flexing to provide comfort during activities of daily living. The inner component provides rigid support to prevent stenosis of the canal and introitus with consistent radial pressure throughout and, given its position within the outer component and middle component, does not touch the healing vaginal tissue. The suturing tab portion provides stability and includes a notch for the tube of a urinary catheter, such as a Foley catheter, to exit the body. The suturing tab portion is removed postoperatively along with the inner component and Foley catheter, typically after about five days.

In use, and after removal of the inner component, a tube attachment of a douche apparatus can be inserted into the open canal of the middle component to fluidly couple the douche apparatus with the outer component at the proximal end of the stent device. The tube attachment interfaces with the outer component via a short internal tube structure and an aperture at the proximal end of the outer component. The aperture is fluidly coupled with a plurality of channels emanating therefrom in an external surface of the outer component to provide even fluid flow over the external surface of the outer component during douching. The number of channels can vary in embodiments, such as at least four channels, at least eight channels, at least twelve channels, or more or fewer channels that may be present in other embodiments. In one embodiment the outer component comprises twelve channels.

In embodiments, the distal end of the stent device comprises a tapered neck having a diameter smaller than a diameter of the body of the stent device. The diameters can be of various dimensions depending on the native anatomy of the cavity created during surgery. This structure conforms to a more natural vaginal canal and introitus.

Embodiments of the flushing vaginal stent device can enable patient discharge from the hospital sooner after a genital surgery than is currently done, as the inner rigid component can be inserted into the stent device post-operatively and removed later during a routine outpatient visit. The patient also no longer has to dilate during the months post-operation as the stent device could remain inside the patient, providing a comfortable and constant dilation effect. The stent device can be replaced once a month with a larger size model, as is currently done with normal dilator practices, simulating the dilating effect without giving the vaginal canal tissue the opportunity to wrinkle, heal closed, or stenose. The stent device also enables the patient to douche through the device more efficiently and effectively on an ongoing basis, replacing the need for a separate dilator and douche devices known conventionally.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1A is a perspective view of a flushing stent device according to an embodiment.

FIG. 1B is a perspective view of a flushing stent device according to an embodiment.

FIG. 1C is a cutaway view of the flushing stent device of FIG. 1B.

FIG. 1D is a cutaway view of the flushing stent device of FIG. 1B.

FIGS. 1E-1K are perspective views of a middle component, inner component and suturing tab portion of a flushing stent device arranged together according to an embodiment.

FIG. 1L is a distal end view of the middle component, inner component and suturing tab portion of FIGS. 1E-1K.

FIG. 1M is a proximal end view of the middle component, inner component and suturing tab portion of FIGS. 1E-1K.

FIG. 1N is a front view of the middle component, inner component and suturing tab portion of FIGS. 1E-1K.

FIG. 1O is a longitudinal cross-sectional view taken along line A-A of FIG. 1N.

FIG. 1P is a side view of the middle component, inner component and suturing tab portion of FIGS. 1E-1K.

FIG. 1Q is a longitudinal cross-sectional view taken along line B-B of FIG. 1P.

FIG. 1R is a front view of the middle component, inner component and suturing tab portion of FIGS. 1E-1K.

FIG. 1S is a lateral cross-sectional view taken along line G-G of FIG. 1R.

FIG. 1T is a detailed view of cross-section H of FIG. 1S.

FIG. 1U is a proximal end view of the middle component, inner component and suturing tab portion of FIGS. 1E-1K.

FIG. 1V is a longitudinal cross-sectional view taken along line E-E of FIG. 1U.

FIG. 1W is a detailed view of partial cross-section F of FIG. 1V.

FIGS. 2A and 2B are perspective views of a middle component and an outer component, respectively, of the flushing stent device of FIG. 1A.

FIG. 2C is a perspective view of the middle component of FIG. 2A arranged within the outer component of FIG. 2B according to an embodiment.

FIG. 2D is a cut-away view of the layers of FIG. 2C.

FIG. 2E is longitudinal cross-sectional view of the layers of FIG. 2C.

FIGS. 2F and 2G are side schematic views of the outer component and the middle component, respectively.

FIG. 3A is a side view of the outer component of the flushing stent device.

FIG. 3B is a perspective view of the outer component of the flushing stent device.

FIG. 3C is another perspective view of the outer component of the flushing stent device.

FIG. 3D is a distal end view of the outer component of the flushing stent device.

FIG. 3E is a proximal end view the outer component of the flushing stent device.

FIG. 3F is another side view of the outer component of the flushing stent device.

FIG. 3G is a longitudinal cross-sectional view taken along line A-A of FIG. 3F.

FIG. 4A is a side view of the middle component of the flushing stent device.

FIG. 4B is a perspective view of the middle component of the flushing stent device.

FIG. 4C is another perspective view of the middle component of the flushing stent device.

FIG. 4D is a distal end view of the middle component of the flushing stent device.

FIG. 4E is a proximal end view the middle component of the flushing stent device.

FIG. 4F is a side view of the middle component of the flushing stent device.

FIG. 4G is a longitudinal cross-section view of the middle component taken along line A-A of FIG. 4F.

FIG. 5A is a perspective view of a radial cam expander of the inner component prior to expansion according to an embodiment.

FIG. 5B is a longitudinal cross-sectional view of FIG. 5A.

FIG. 5C is a perspective view of a radial cam expander of the inner component after expansion according to an embodiment.

FIG. 5D is a longitudinal cross-sectional view of FIG. 5C.

FIG. 5E is an oblique view of a radial cam expander of the inner component after expansion according to an embodiment.

FIG. 5F is a longitudinal cross-sectional view taken along line C-C of FIG. 5E

FIG. 5G is a detailed view of partial cross-section D of FIG. 5F.

FIG. 5H is a perspective view of a radial cam expander of the inner component according to an embodiment.

FIG. 5I is a detailed view of partial perspective section M of FIG. 5H.

FIG. 5J is a perspective view of a radial cam expander of the inner component after expansion according to an embodiment.

FIG. 5K is a detailed lateral cross-sectional view taken along like O-O of FIG. 5J.

FIG. 5L is a perspective view of a radial cam of an inner component according to an embodiment.

FIG. 6A is a perspective view of the outer component according to an embodiment.

FIG. 6B is a perspective view of the outer component according to an embodiment.

FIG. 6C is a perspective view the outer component according to the embodiment of FIG. 6A.

FIG. 6D is a perspective view of the outer component according to the embodiment of FIG. 6B.

FIG. 7 is a perspective view of the middle component according to an embodiment.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

Embodiments relate to flushing vaginal stent devices for supporting, and preventing stenosis and infection of, a vagina after genital surgery or radiation therapy.

In general throughout this document, and unless otherwise noted, “proximal” is used to refer to an end or portion that is closest to the center of mass (abdomen) of the body (i.e. the device end or portion that in situ is closest to the center of mass of the body), and “distal” is used to refer to an end or portion that is furthest from the center of mass (abdomen) of the body (i.e. device end or portion that in situ is furthest to the center of mass of the body, which is also describing the vaginal opening end).

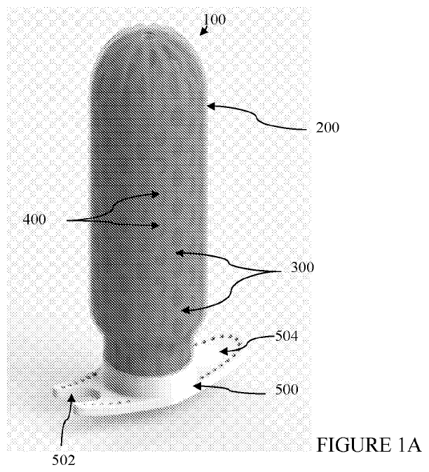

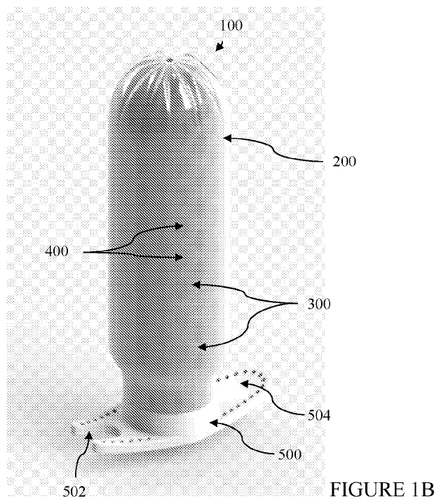

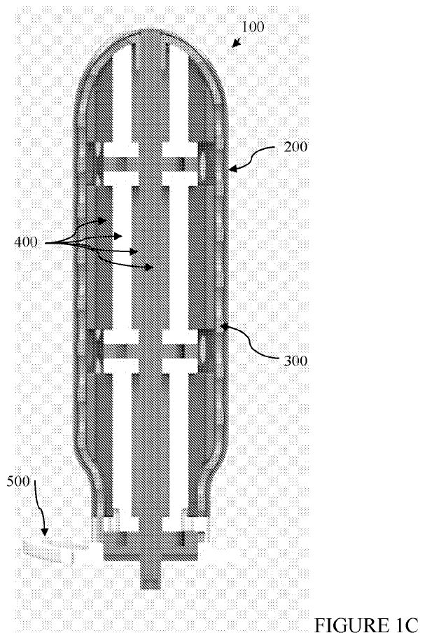

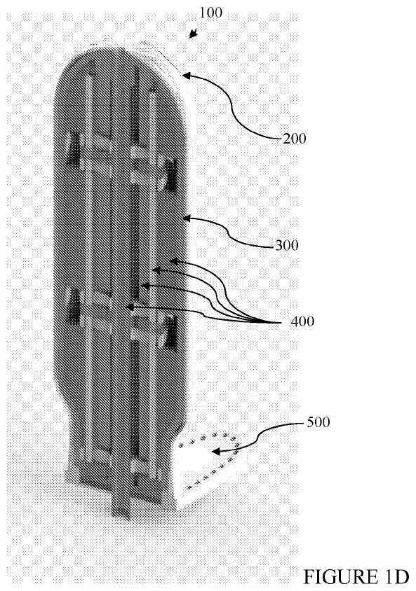

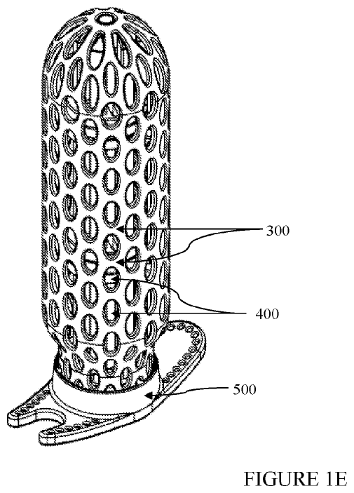

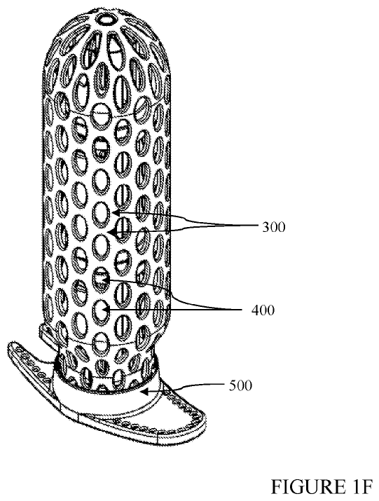

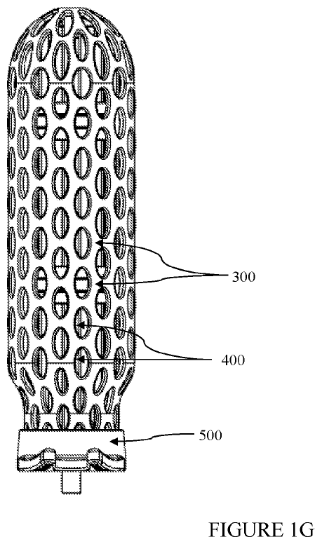

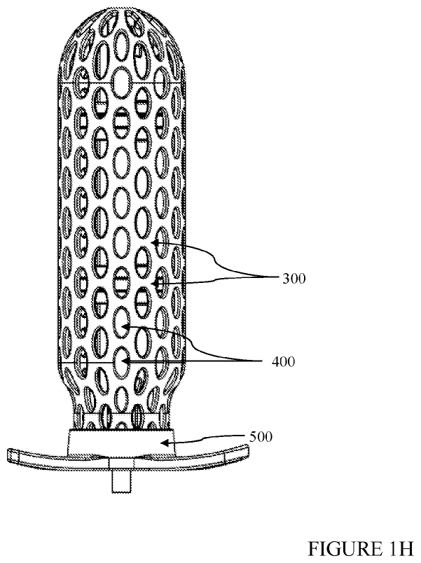

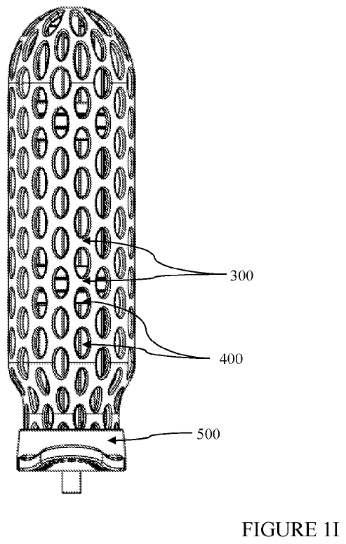

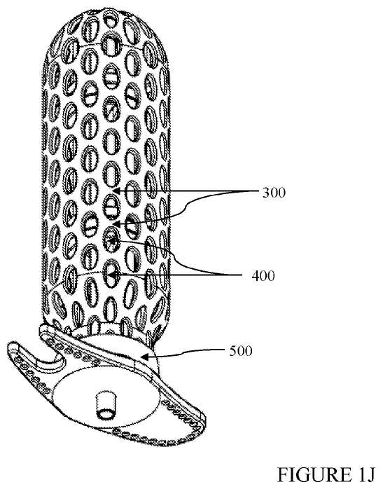

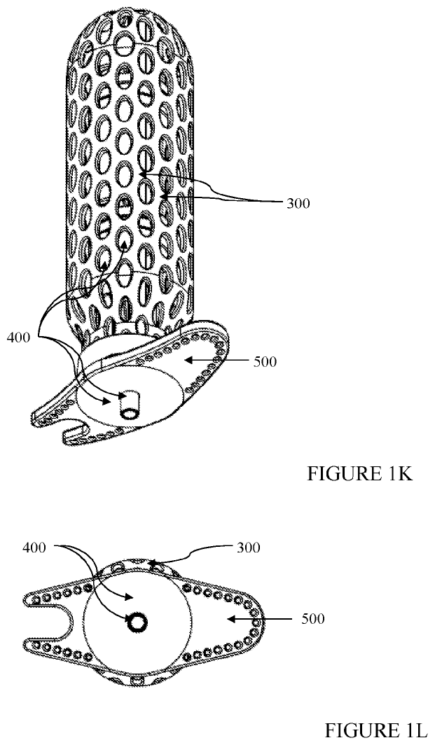

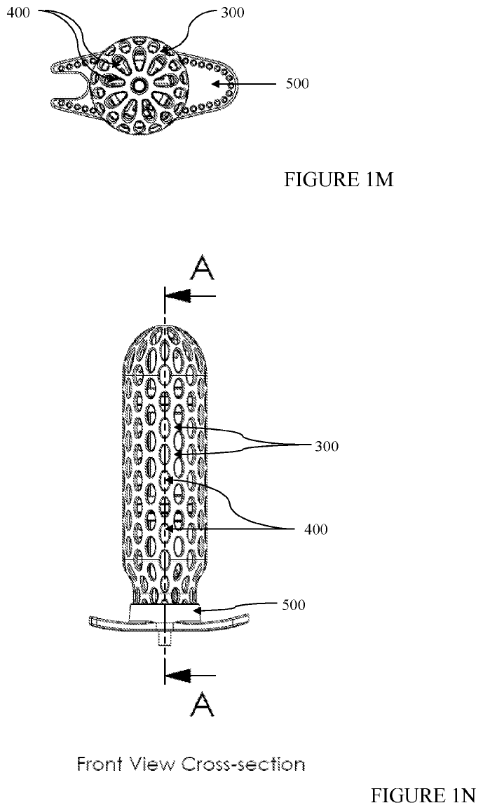

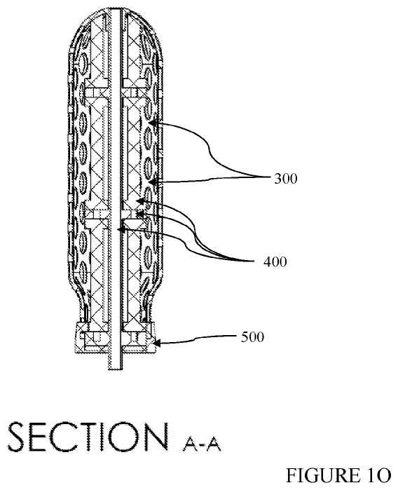

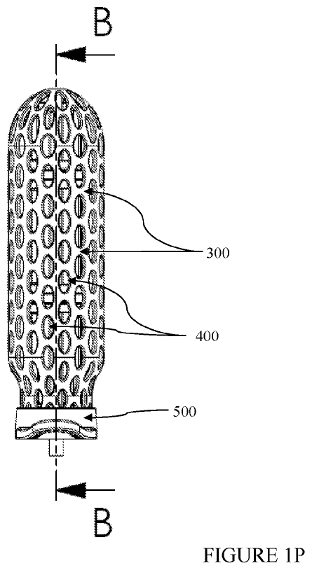

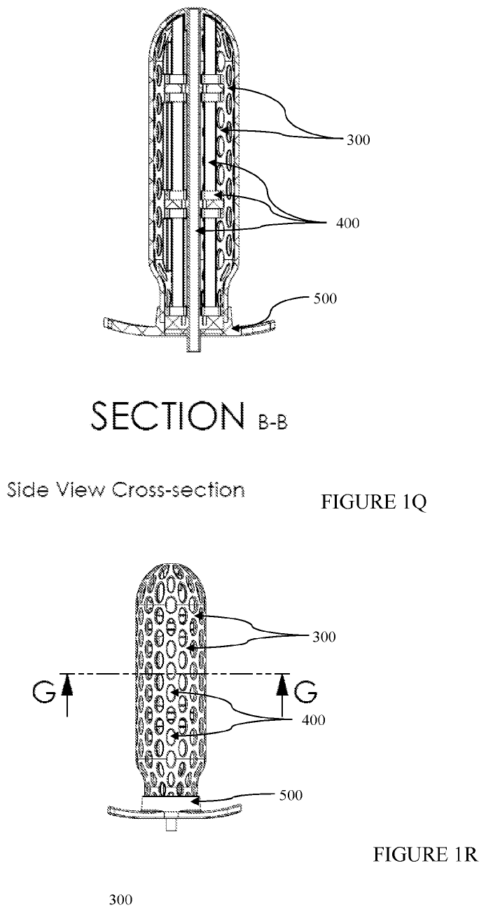

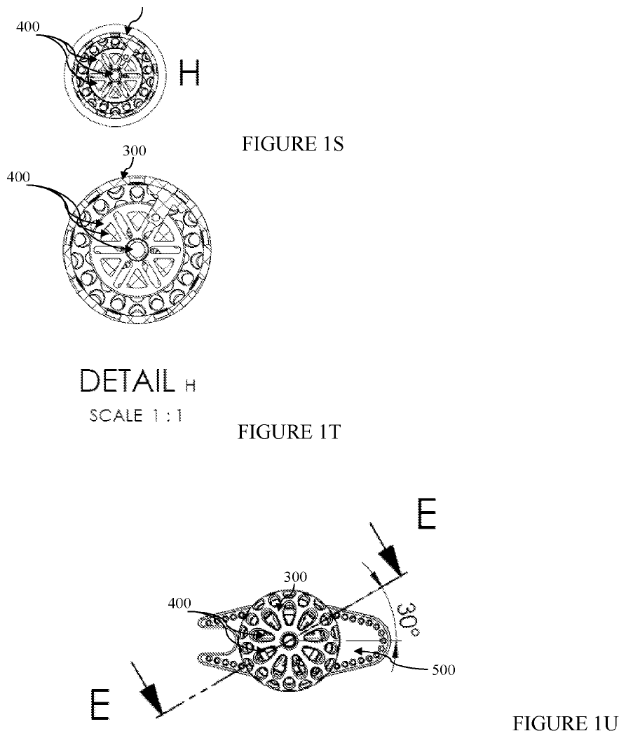

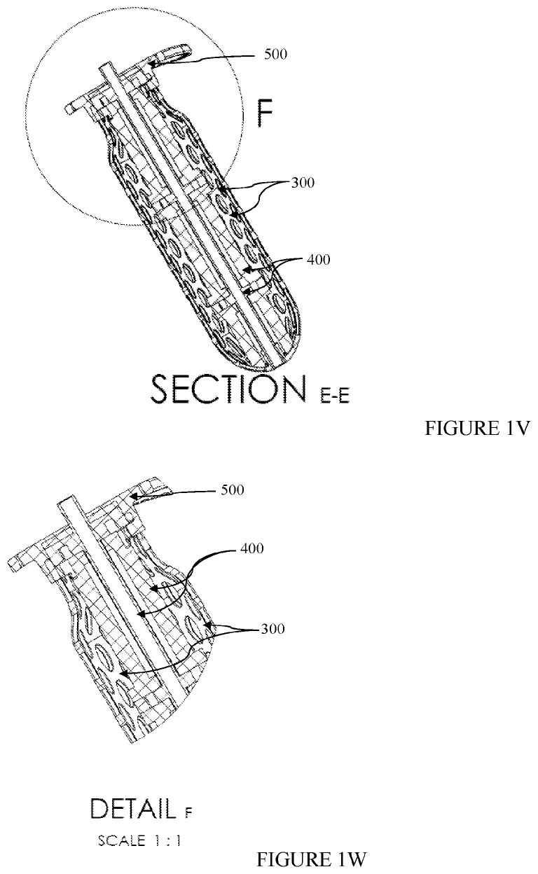

FIGS. 1A and 1B are perspective views of a flushing stent device 100 according to an embodiment. Referring also to FIGS. 1C and 1D, flushing stent device 100 comprises an outer component 200, a middle component 300 configured to be arranged within outer component 200, and an inner component 400 configured to be arranged within middle component 300. In some embodiments, inner component 400 comprises a suturing tab portion 500 that is arranged external to flushing stent device 100 at a distal end when inner component 400 is arranged within middle component 300 and outer component 200. The proximal end of flushing stent device 100 is generally rounded or domed, and the distal end of flushing stent device 100 is open.

FIGS. 1E-1K are perspective views of middle component 300, inner component 400 and suturing tab portion 500 arranged together according to an embodiment. FIG. 1L is a distal end view and FIG. 1M is a proximal end view of middle component 300, inner component 400 and suturing tab component 500 of FIGS. 1E-1K. FIGS. 1N-1W provide additional perspective and cross-sectional views of middle component 300, inner component 400 and suturing tab portion 500 of FIGS. 1E-1K.

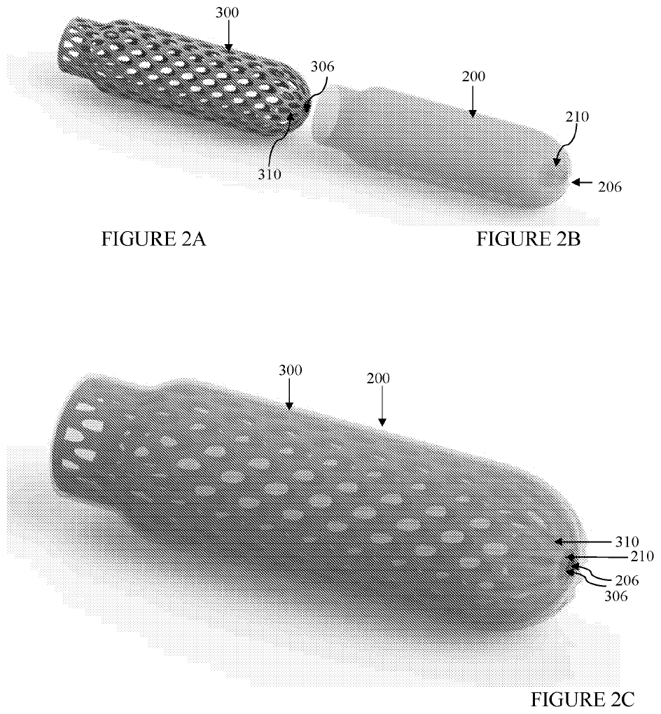

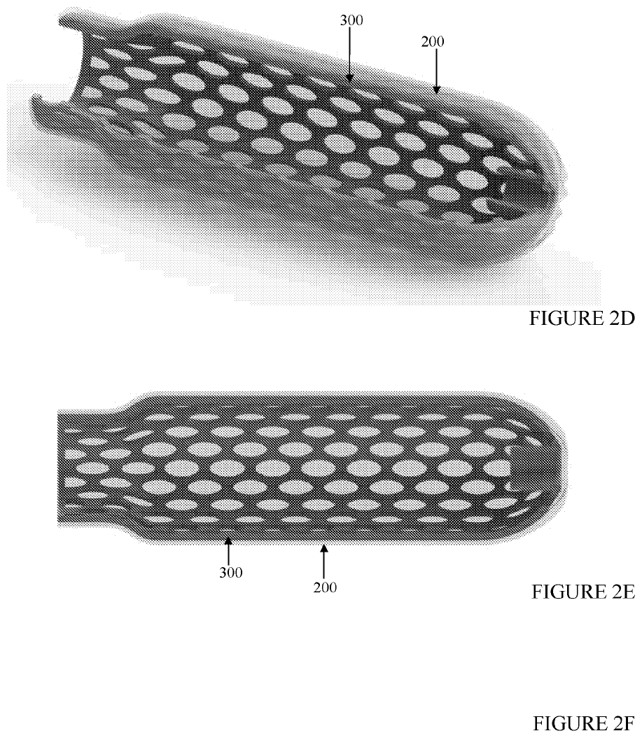

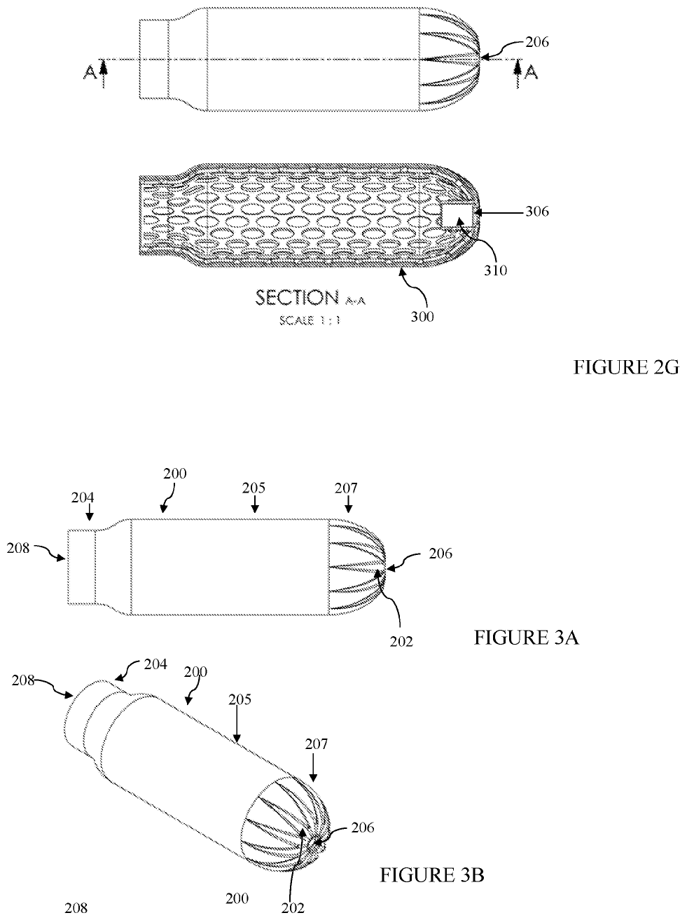

FIGS. 2A and 2B depict middle component 300 and outer component 200, respectively. Outer component 200 comprises an aperture 206 fluidly coupled with a tube structure 210 at the proximal end. Aperture 206 is aligned with a corresponding aperture 306 on middle component 300 when middle component 300 is arranged within outer component 200, which can be seen in FIGS. 2C and 2D.

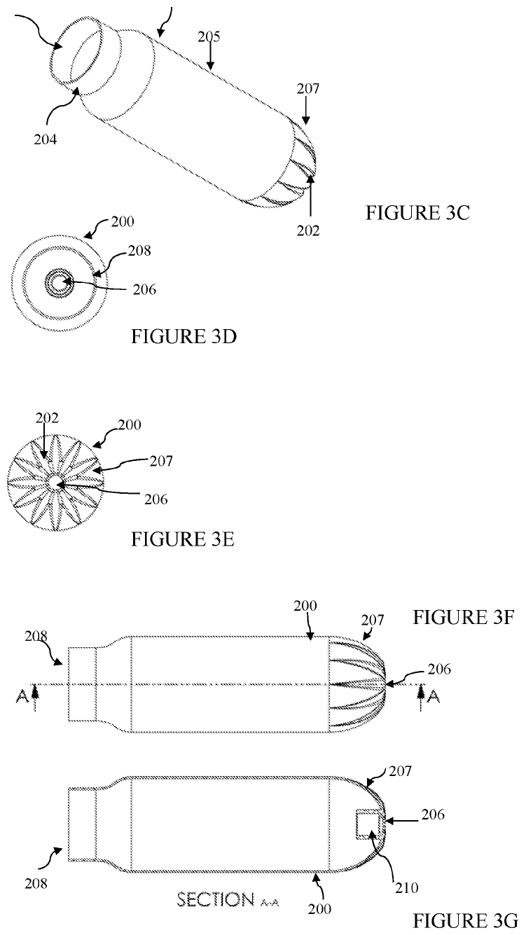

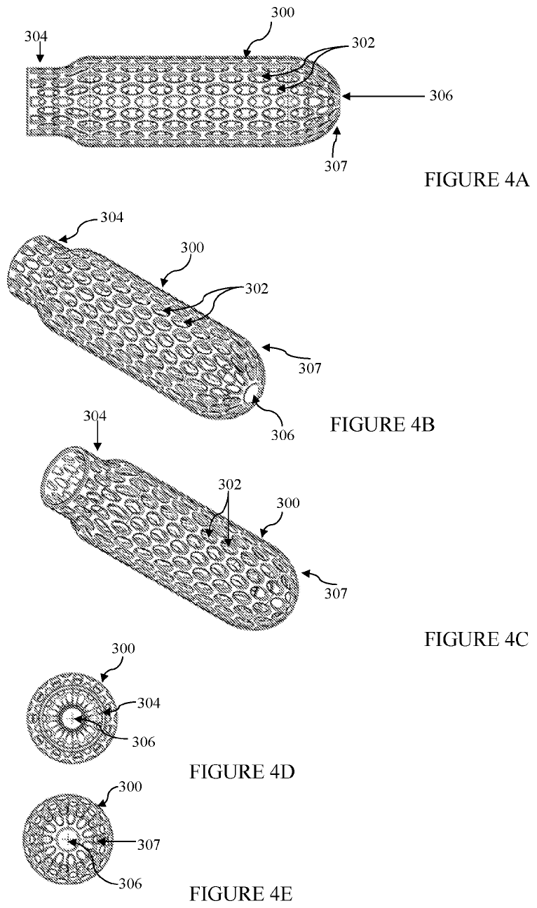

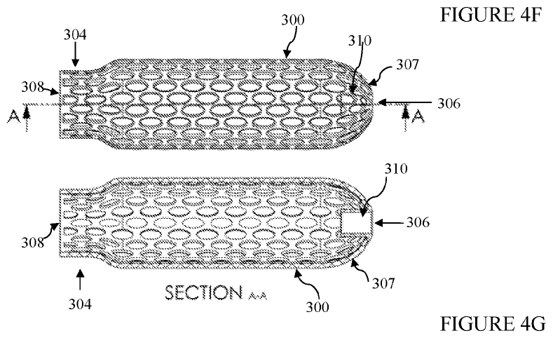

As can be seen in FIGS. 2F, 3A, 3B, 3C, 3E and 3F, outer component 200 also comprises a plurality of external channels 202 mechanically and fluidly coupled with aperture 206 and tube structure 210. Channels 202 extend or radiate away from aperture 206 and partially down the sides of outer component 200. In the embodiment depicted, channels 202 are tapered and extend along a cap section 207 of outer component 200, though in other embodiments channels 202 may extend father or less far along outer component 200. Channels 202 also can be deeper, shallower, or configured in ways other than those specifically depicted in the examples of the drawings, as appreciated by those of ordinary skill in the art. In general, channels 202 are configured to provide even fluid distribution during douching when a douche apparatus is inserted into flushing stent device 100 and mechanically and fluidly coupled with tube structure 210, which helps minimize the risk of tissue in-growth along flushing stent device 100, as douching is used both to heal tissue and to prevent adhesion of tissue to outer component 200. Outer component 200 also includes a distal end 208 that is aligned with a corresponding distal end 308 on middle component 300 when middle component 300 is arranged within outer component 200 (see FIGS. 3F, 3G, and 4G). Distal end 208 and distal end 308 are each open.

Outer component 200 also comprises an introital portion 204, which has a smaller diameter than a shaft portion 205 of outer component 200. In one embodiment, introital portion 204 tapers to approximately three centimeters in diameter, and shaft portion 205 is approximately four centimeters in diameter. These dimensions are exemplary of only one embodiment, and in other embodiments the diameters may be smaller or larger or may have different relative diameters (i.e., the diameter of introital section 204 may be more or less than 1 cm different from a diameter of shaft section 205)

A length of outer component 200 in various embodiments is in a range of about 10 cm to about 20 cm, such as about 10 cm to about 15 cm. In other embodiments, the length of outer component 200 may be shorter or longer, as may relate to the natural anatomy in some women. While flushing stent device 100 is depicted herein as being generally straight, in other embodiments flushing stent device 100 (or one or more layers thereof, such as middle component 300) can be curved or include a curved section or portion. For example, in some embodiments a proximal third or distal third of flushing stent device 100 can have a slight angle of curvature, which can mimic a more natural anatomy in some women.

In various embodiments, outer component 200 is approximately 0.1-4 millimeters thick and is flexible. Outer component 200 comprises a biocompatible material in embodiments and is suitable for use with various douching fluids, including povidone-iodine douching fluid, and does not decompose or leech out into the surrounding body tissue. Outer component 200 also prevents the surrounding body tissue from healing or growing into flushing stent device 100, particularly the fenestrated structure of middle component 300.

Referring to FIGS. 2A, 2C, 2D, 2E, 2G and 4A-4G, middle component 300 is configured to fit snugly within outer component 200, though some relative movement of the components 200, 300 may occur in use. Middle component 300 comprises a porous, fenestrated, or lattice structure configured to provide support to flushing stent device 100. The size and shape of apertures 302 (i.e., the fenestrations) can vary in embodiments and can be round, ovular, hexagonal, square- or diamond-shaped, or have some other suitable shape. In some embodiments, the size or shape (or both) of the fenestrations varies on different sections of middle component 300 (i.e., distal introital section 304, main shaft section 305, proximal cap section 307), for example to provide different mechanical properties or levels of support. In general, the fenestrated structure of middle component 300 is configured to be flexible but strong enough to provide sufficient radial pressure throughout middle component 300 (and thereby flushing stent device 100) to prevent post-surgical complications such as stenosis.

Middle component 300, like outer component 200, also comprises an aperture 306 at the proximal end or cap section 307. This allows a douching tube (not shown in the drawings) to be inserted through middle component 300 from the distal end to mechanically and fluidly couple with flushing stent device 100 for douching from the proximal end.

Similar to outer component 200, middle component 300 includes an introital section 304 having a smaller diameter than a body portion. The length of middle component 300 is similar to outer component 200, approximately 10 cm to 20 cm, for example 10 cm to 15 cm, though the length of middle component 300 may be slightly less than the length of outer component 200 so that middle component 300 can fit within outer component 200. In other embodiments, the length of middle component 300 may be shorter or longer, depending on the length of outer component 200 and for the natural anatomy in some women. Again, similar to outer component 200, middle component 300 also can be curved or include one or more curved portions.

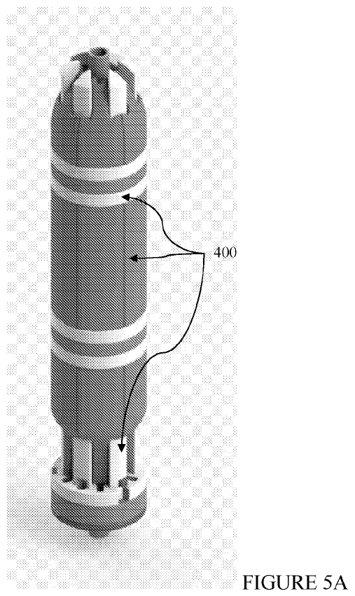

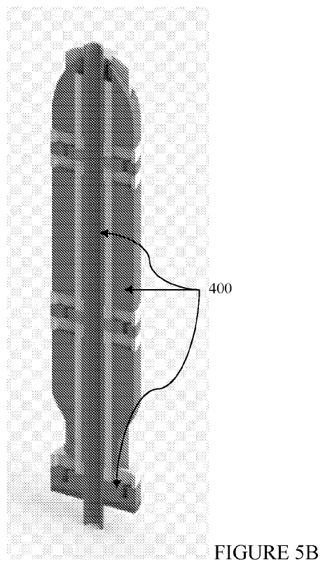

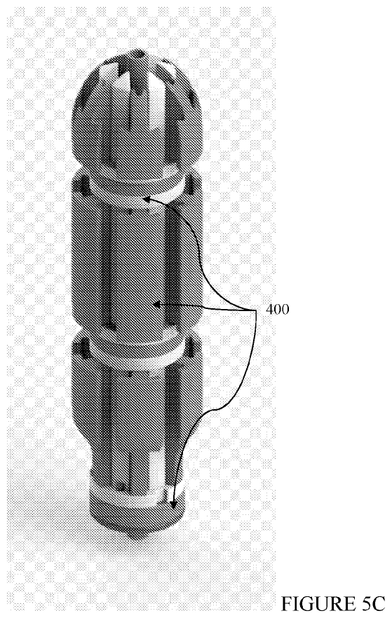

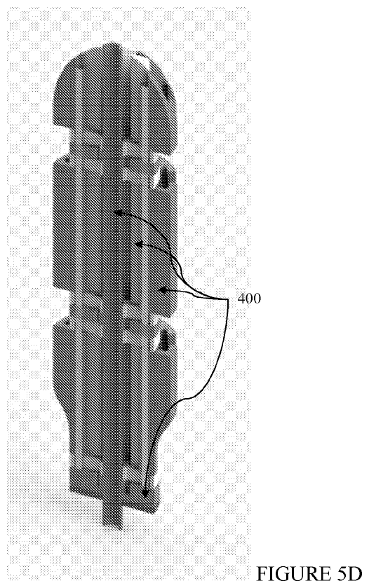

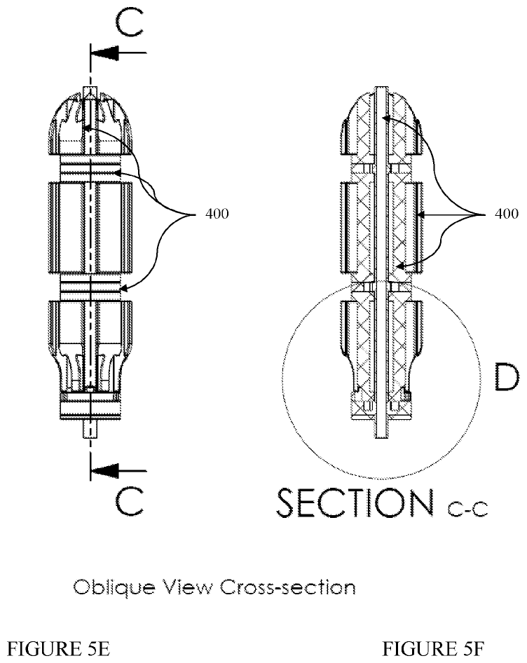

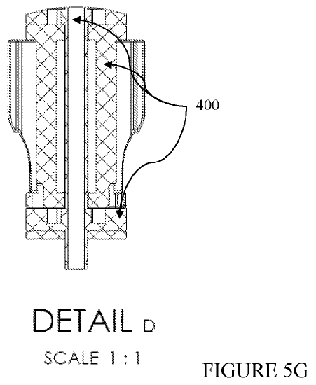

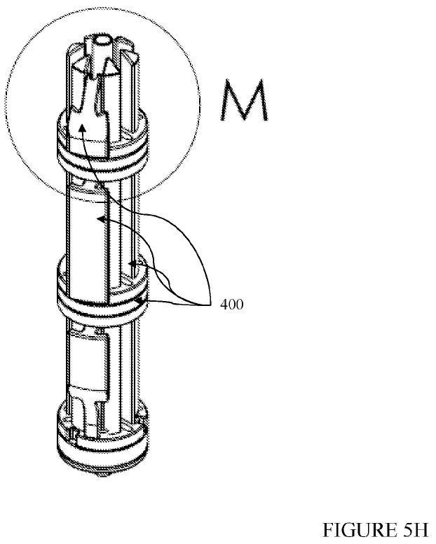

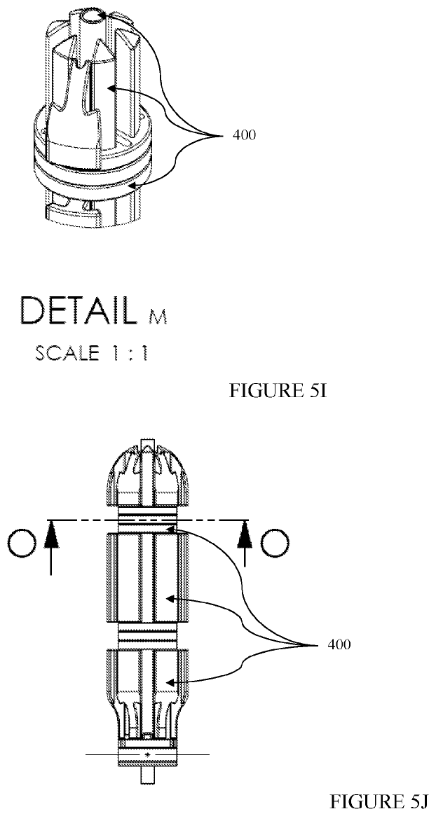

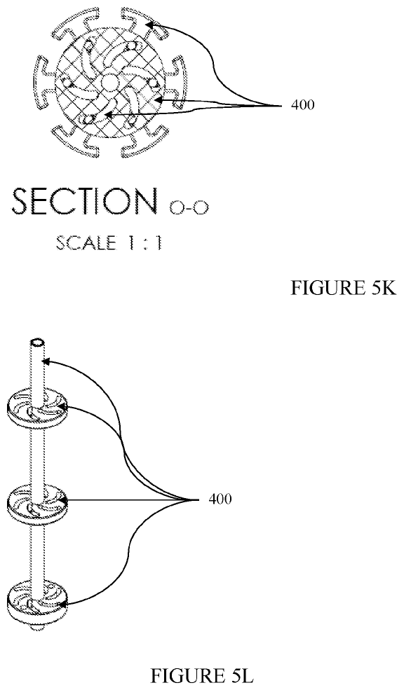

Referring generally to FIGS. 5A-5L, inner component 400 is configured to fit within middle component 300, though some relative movement of the components 400, 300 may occur in use. In some embodiments (as discussed below), inner component 400 may be inserted or provided in a first retracted configuration and later transitioned by a physician to a second expanded configuration in situ. Inner component 400 may later be transitioned back to the first retracted configuration for removal after the initial post-operative period (e.g., five days). In other embodiments, inner component 400 may have a single or static configuration or may be removed from within middle component 300 in either the first or second configurations. Inner component 400 is open or comprises an aperture at its distal end to receive surgical packing materials like gauze or other materials in post-operative use.

Similar to outer component 200 and middle component 300, inner component 400 can comprise an introital section having a smaller diameter than a body portion. The length of inner component 400 is similar to outer component 200 and middle component, approximately 10 cm to 20 cm, for example 10 cm to 15 cm, though the length of inner component 400 may be slightly less than the length of middle component 300 so that inner component 400 can fit within middle component 300. In other embodiments, the length of inner component 400 may be shorter or longer, depending on the length of middle component 300 and for the natural anatomy in some women. Again, similar to outer component 200 and middle component 300, some embodiments of inner component 400 can be curved or include one or more curved portions.

In some embodiments inner component 400 comprises or is a radial cam expander. In these embodiments, the radial cam expander is configured to transition inner component 400 from the first relaxed configuration (e.g., as shown in FIGS. 5A and 5B) to the second expanded configuration (e.g., as shown in FIGS. 5C and 5D). Thus, in one embodiment the radial cam expander comprises a central shaft running longitudinally (i.e., parallel to a long axis of flushing stent device 100 extending between the distal end and the proximal end), a plurality of radial members each having a cam follower; at least one guide plate comprising a plurality of channels; at least one cam plate including a plurality of internal cam profiles such that the cam followers fixed on the radial members interface with the cam plate; and a rotational lock plate configured as a key to fix the radial cam expander in the radially expanded or retracted state.

In other embodiments, other radial cam expander components or configurations can be used. In still other embodiments, flushing stent device 100 comprises another type of expansion/retraction mechanism configured to transition inner component 400 or flushing stent device 100 more generally between a retracted configuration and an expanded configuration. Similar to a speculum or other medical device (though the particular expansion/retraction mechanism may vary from a speculum), the expansion/retraction mechanism enables inner component 400 or flushing stent device 100 to be inserted by a physician in a retracted configuration and transitioned in situ to an expanded configuration.

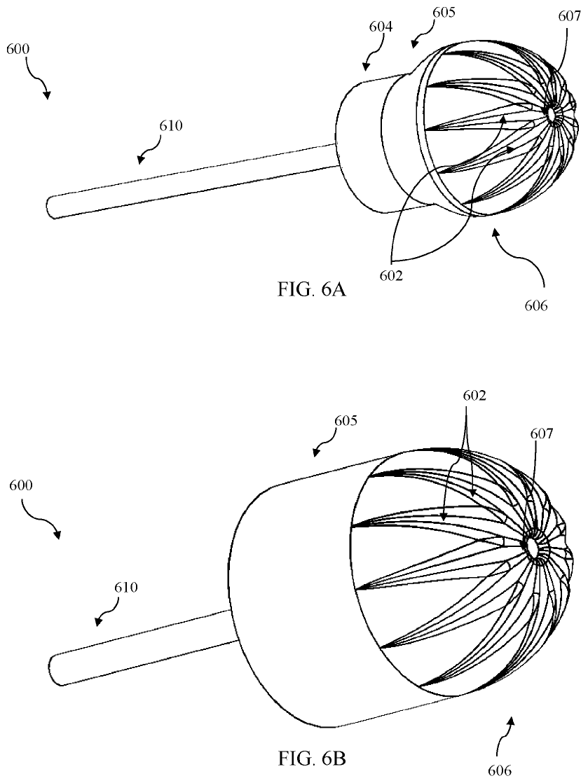

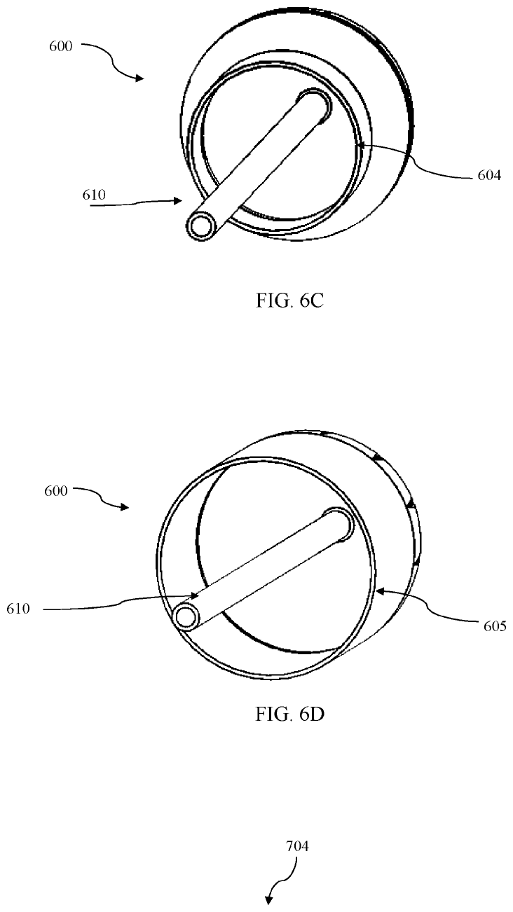

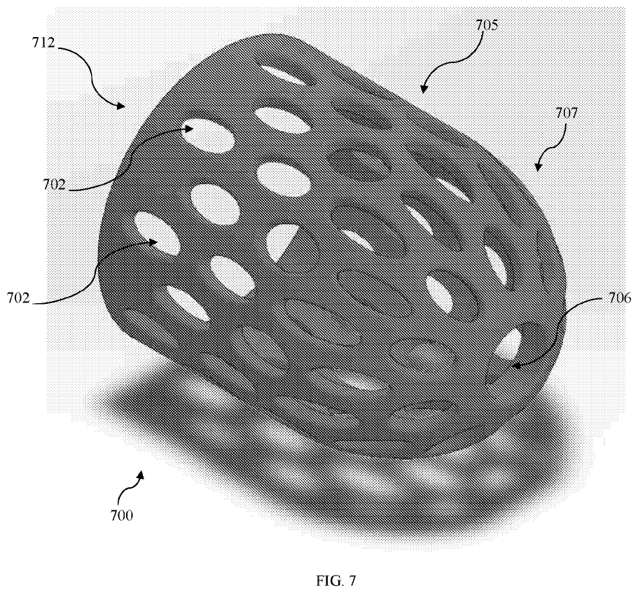

FIGS. 6A and 6B depict another embodiment of an outer component 600 for a flushing stent device. Outer component 600 generally comprises a proximal cap section 607 having a main shaft section 605 and an aperture 606 mechanically and fluidly coupled with a tube structure assembly 610. Aperture 606 and tube structure 610 are functionally similar to aperture 206 and tube structure 210 of outer component 200. Generally, outer component 600 is formed from materials analogous to the materials forming outer component 200.

Proximal cap section 607 also comprises a plurality of external channels 602 mechanically and fluidly coupled with aperture 606 and tube structure 610. Channels 602 are analogous to channels 202, described above. In general, channels 602 are configured to provide even fluid distribution during douching when a douche apparatus is inserted mechanically and fluidly coupled with tube structure 610, which helps minimize the risk of tissue in-growth along outer component 620, as douching is used both to heal tissue and to prevent adhesion of tissue to outer component 620.

Some embodiments of outer component 600 include an introital portion 604 at the distal end of shaft section 605, as shown in FIGS. 6A and 6C. Introital portion 604 has a smaller diameter than proximal cap section 607. In these embodiments, main shaft section 605 may also be tapered, according to the difference in diameter between introital portion 604 and proximal cap section 607. In other embodiments, for example as depicted by FIGS. 6B and 6D, outer component has a generally uniform diameter along the length of shaft section 605.

Embodiments of outer component 600 are configured for use with embodiments of a middle component 700, for example as depicted by FIG. 7. Middle component 700 is structurally analogous to middle component 300, except that the dimensions of middle component 700 are configured according to the dimensions of outer component 600. In particular, this embodiment of flushing stent device 100 is designed to sit above the pelvic floor musculature of a patient and may be scaled as appropriate for either pediatric or adult applications.

The length and dimensions of middle component 700 are similar to outer component 600, though the length of middle component 700 may be slightly less than the length of outer component 600 so that middle component 700 can fit within outer component 600. In other embodiments, the length of middle component 600 may be smaller or larger, depending on the length of outer component 700 and the natural anatomy in some patients. Again, similar to outer component 700, middle component 600 also can be curved or include one or more curved portions.

Generally, middle component 700 comprises a proximal cap section 707 having an aperture 706 configured for mechanical and fluid connection with tube structure 610, a main shaft section 705, and a distal section 704. Middle component 700 is configured to fit snugly within outer component 600, though some relative movement of the components 600, 700 may occur in use.

Aperture 706 at the proximal cap section 707 allows a douching tube (not shown in the drawings) to be inserted through middle component 700 from the distal end to mechanically and fluidly couple with flushing stent device 100 for douching from the proximal end. For example, the douching tube may be inserted through tube structure 610, which extends past distal section 704 through the interior of main shaft 705.

Embodiments of main shaft 705 include a lattice-like arrangement of apertures 702. Apertures 702 may comprise a uniform arrangement over the entirety of main shaft 705, as depicted in FIG. 7. Alternatively, apertures 702 may only cover certain portions of main shaft 705. The size and shape of apertures 702 can vary in embodiments and can be round, ovular, hexagonal, square-or diamond-shaped, or have some other suitable shape. In some embodiments, the size or shape (or both) of the fenestrations varies on different sections of middle component 700 (i.e., distal section 704, main shaft section 705, proximal cap section 707), for example to provide different mechanical properties or levels of support. In general, the fenestrated structure of middle component 700 is configured to be flexible but strong enough to provide sufficient radial pressure throughout middle component 700 (and thereby flushing stent device 100) to prevent post-surgical complications such as stenosis.

Distal section 704 is open at the distal end, providing access to an interior cavity 712. In some embodiments, interior cavity 712 is configured to receive surgical packing materials like gauze or other materials in post-operative use. In alternative embodiments, interior cavity 712 is configured to receive, for example, an expansion and retraction mechanism analogous to inner component 400.

In embodiments, inner component 400 comprises or is configured to temporarily or permanently coupled with suturing tab portion 500. In still other embodiments, suturing tab portion 500 can be configured to temporarily couple or interact with outer component 200, 600 and/or middle component 300, 700. Suturing tab portion 500 comprises a first notched end 502 and a second end 504. Notched end 502 is configured to removably receive a portion of a Foley catheter in some embodiments, which can be used post-operatively to allow for urination. In other embodiments, the notch can be omitted and first end 502 and second end 504 can be similarly or identically configured.

In use, suturing tab portion 500 is configured to remain outside a patient's body when flushing stent device 100 is in situ. Suturing tab portion 500 can assist a physician in inserting and positioning flushing stent device 100, manipulating flushing stent device 100 when in use (i.e., moving flushing stent device 100 to prevent tissue adhesion), and/or assisting in removing inner component 400 post-operatively when it is no longer needed. Suturing tab portion 500 is configured to be positioned such that first notched end 502 is anterior and can be sutured to the patient's vulva. Second end 504 is positioned posterior and can be sutured to the patient's perineum. To facilitate suturing, some embodiments of suturing tab portion 500 comprise a plurality of suturing apertures arranged proximate an edge of suturing tab portion. The size, placement or relative arrangement of the suturing apertures can vary from those depicted in the drawings. In an alternative embodiment, the suturing apertures can be placed on the distal surface of suturing tab portion 500 with eyelets or hoops for sutures to be threaded across suturing tab portion 500 and sutured to the medial aspects of the thigh in the groin region.

In use, flushing stent device 100 is inserted by a physician or a patient into a neovagina or post-operative vagina. When inserted, flushing stent device 100 can comprise two components (i.e., outer component 200, 600 and middle component 300, 700, with inner component 400 and suturing tab portion 500 omitted or inserted separately) or three components (i.e., all of outer component 200, 600, middle component 300, 700, and inner component 400 with or without suturing tab portion 500 preassembled). Once positioned, and if applicable, the physician can expand inner component (in embodiments in which inner component 400 is configured to have both retracted and expanded states) and suture flushing stent device 100 into place via suturing tab portion 500 and using suturing materials (e.g., needle, sutures). Inner component 400 can be pre-packed with surgical packing materials before insertion, or surgical packing materials can be inserted once inner component 400 is in situ (and optionally expanded, in embodiments in which expansion/retraction is used). If inner component 400 is omitted, the surgical packing materials may be pre-packaged or inserted within interior cavity 712. After some post-operative period (such as about 5 days), the physician can remove the materials or components within interior cavity 712, such as inner component 400 and suturing tab component 500, leaving outer component 200, 600 and middle component 300, 700 of flushing stent device 100 in situ. Outer component 200, 600 and middle component 300, 700 of flushing stent device 100 can remain in place for weeks or months, and the aperture/tube assembly structure enables the patient to douche whenever needed by inserting a tube of an external douche assembly into middle component 300, 700 of flushing stent device and couple at the proximal end of the tube to tube assembly 310, 610. In use, douching fluid can be provided to an external surface of flushing stent device 100 via apertures 306, 706 and 206, 606 and channels 202, 602.

In some embodiments, flushing stent device 100 can be provided in or with a kit offered for sale. The kit can comprise at least one flushing stent device 100, and in some embodiments comprises a plurality of flushing stent devices 100, such as different sizes of flushing stent devices 100. The one or more flushing stent devices 100 can be provided in sterile packaging, and the kit also can include one or more instructions for use, surgical packing materials, a Foley or other catheter device, a douching apparatus or fluid, sutures or materials for suturing, or other components configured to support physician understanding and use of flushing stent device 100 or patient post-operative care or interaction with flushing stent device 100 in situ.

Various advantages are provided by embodiments of the flushing stent device disclosed herein when compared with conventional approaches, including reduced hospitalization times, more patient comfort and control, and the ability to douche multiple times daily without having to remove or replace the flushing stent device.

In an example embodiment, a flushing vaginal stent device comprises an outer component having an outer component proximal end and an outer component distal end, the outer component proximal end comprising a tube portion fluidly coupled with an aperture that is fluidly coupled with a plurality of channels formed in an outer surface of the outer component, and the outer component distal end being open; a middle component having a middle component proximal end with an aperture and a middle component distal end and comprising a fenestrated body structure between the middle component proximal end and the middle component distal end, the middle component configured to be arranged within the outer component such that the aperture of the middle component proximal end and the aperture of the outer component proximal end are arranged together and the outer component proximal end and the middle component proximal end are arranged together and the outer component distal end and the middle component distal end are arranged together; and an interior component having an interior proximal end and an interior distal end, the interior component configured to be arranged within the middle component such that the outer component proximal end, the middle component proximal end, and the interior component proximal end are arranged together and the outer component distal end, the middle component distal end, and the interior component distal end are arranged together.

The interior component can further comprise a mechanism configured to move the interior component between a retracted state and an expanded state.

The flushing vaginal stent device can further comprise a suturing tab portion having a first end and a second end, the suturing tab portion coupled with the mechanism.

The suturing tab portion can be configured to temporarily couple with at least one of the outer component or the middle component.

The suturing tab portion can be permanently coupled with the mechanism and the mechanism can be removably arranged within the interior component such that the suturing tab portion and the mechanism can be removed simultaneously without removing the outer component or the middle component.

The suturing tab portion can further comprise a catheter aperture formed in the first end.

The suturing tab portion can further comprise a plurality of suturing tab apertures arranged proximate an edge of the suturing tab portion.

The internal tube portion can extend past the outer component distal end, the middle component distal end, and the interior distal end.

At least one of a length of each of the outer component, the middle component, and the interior component, or a diameter of each of the outer component, the middle component, and the interior, can be configured for placement in a pediatric patient.

The outer component distal end, the middle component distal end, and the interior distal end can further comprise a tapered introital section reducing a diameter of the outer component distal end, a diameter of the middle component distal end, and a diameter the interior distal end.

Each of the plurality of channels can radiate away from the aperture and extend partially down sides of the outer component.

In an example embodiment, a kit for vaginal flushing comprises at least one flushing stent device as described in any of the preceding paragraphs, and at least one of: instructions for use; sterile packaging; surgical packing material; catheter; douche device; catheter tubing; or suturing material.

In an example embodiment, a method comprises providing a flushing vaginal stent device comprising: an outer component having an outer component proximal end and an outer component distal end, the outer component proximal end comprising a tube portion fluidly coupled with an aperture that is fluidly coupled with a plurality of channels formed in an outer surface of the outer component, and the outer component distal end being open; a middle component having a middle component proximal end with an aperture and a middle component distal end and comprising a fenestrated body structure between the middle component proximal end and the middle component distal end, the middle component configured to be arranged within the outer component such that the aperture of the middle component proximal end and the aperture of the outer component proximal end are arranged together and the outer component proximal end and the middle component proximal end are arranged together and the outer component distal end and the middle component distal end are arranged together; and an interior component having an interior proximal end and an interior distal end, the interior component configured to be arranged within the middle component such that the outer component proximal end, the middle component proximal end, and the interior component proximal end are arranged together and the outer component distal end, the middle component distal end, and the interior component distal end are arranged together.

The method can further comprise providing an expansion and retraction mechanism removably coupled to the interior component to facilitate expanding a volume of the interior component.

The method can further comprise facilitating retraction and removal of the expansion and retraction mechanism and the surgical packing material after a post-operative period.

The method can further comprise configuring the middle component for: insertion of a douche device into the middle component in situ, coupling of the douche device to the tube portion, and expressing of douche fluid from the douche device, through the tube portion, through the proximal end aperture, and into the plurality of channels.

The method can further comprise configuring the proximal end aperture and the plurality of channels such that douche fluid is provided to an external surface of the outer component.

The method can further comprise providing a suturing tab portion coupled with at least the interior component to facilitate selective securing of the flushing stent device to the patient by suturing the flushing stent device to the patient.

The method can further comprise configuring the suturing tab portion for selective removable coupling of a catheter to a notched end of the suturing tab portion.

In embodiments of the method, the suturing portion can further comprise a plurality of suturing tab apertures arranged proximate an edge of the suturing tab portion to facilitate the selective securing of the flushing stent device to a patient by applying sutures to the suturing tab portion.

The method can further comprise configuring the tube position to extend past the distal end such that the tube portion is accessible via a vaginal canal of the patient when the flushing stent device is in situ above a pelvic floor musculature of the patient.

The method can further comprise extending the tube portion.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1. A flushing vaginal stent device comprising:
an outer component having an outer component proximal end and an outer component distal end, the outer component proximal end comprising a tube portion fluidly coupled with an aperture that is fluidly coupled with a plurality of channels formed in an outer surface of the outer component, and the outer component distal end being open;
a middle component having a middle component proximal end with an aperture and a middle component distal end and comprising a fenestrated body structure between the middle component proximal end and the middle component distal end, the middle component configured to be arranged within the outer component such that the aperture of the middle component proximal end and the aperture of the outer component proximal end are arranged together and the outer component proximal end and the middle component proximal end are arranged together and the outer component distal end and the middle component distal end are arranged together; and
an interior component having an interior proximal end and an interior distal end, the interior component configured to be arranged within the middle component such that the outer component proximal end, the middle component proximal end, and the interior component proximal end are arranged together and the outer component distal end, the middle component distal end, and the interior component distal end are arranged together.

2. The flushing vaginal stent device of claim 1, wherein the interior component further comprises a mechanism configured to move the interior component between a retracted state and an expanded state.

3. The flushing vaginal stent device of claim 2, further comprising a suturing tab portion having a first end and a second end, the suturing tab portion coupled with the mechanism.

4. The flushing vaginal stent device of claim 3, wherein the suturing tab portion is configured to temporarily couple with at least one of the outer component or the middle component.

5. The flushing vaginal stent device of claim 3, wherein the suturing tab portion is permanently coupled with the mechanism and the mechanism is removably arranged within the interior component such that the suturing tab portion and the mechanism are removed simultaneously without removing the outer component or the middle component.

6. The flushing vaginal stent device of claim 3, the suturing tab portion further comprising a catheter aperture formed in the first end.

7. The flushing vaginal stent device of claim 3, the suturing tab portion further comprising a plurality of suturing tab apertures arranged proximate an edge of the suturing tab portion.

8. The flushing vaginal stent device of claim 1, wherein the internal tube portion extends past the outer component distal end, the middle component distal end, and the interior distal end.

9. The flushing vaginal stent device of claim 8, wherein at least one of a length of each of the outer component, the middle component, and the interior component, or a diameter of each of the outer component, the middle component, and the interior, are configured for placement in a pediatric patient.

10. The flushing vaginal stent device of claim 1, the outer component distal end, the middle component distal end, and the interior distal end further comprising a tapered introital section reducing a diameter of the outer component distal end, a diameter of the middle component distal end, and a diameter the interior distal end.

11. The flushing vaginal stent device of claim 1, wherein each of the plurality of channels radiates away from the aperture and extends partially down sides of the outer component.

12. A kit for vaginal flushing comprising:
at least one flushing stent device as claimed in claim 1; and
at least one of:
instructions for use;
sterile packaging;
surgical packing material;
catheter;
douche device;
catheter tubing; or
suturing material.

13. A method comprising:
providing a flushing vaginal stent device comprising:
an outer component having an outer component proximal end and an outer component distal end, the outer component proximal end comprising a tube portion fluidly coupled with an aperture that is fluidly coupled with a plurality of channels formed in an outer surface of the outer component, and the outer component distal end being open;
a middle component having a middle component proximal end with an aperture and a middle component distal end and comprising a fenestrated body structure between the middle component proximal end and the middle component distal end, the middle component configured to be arranged within the outer component such that the aperture of the middle component proximal end and the aperture of the outer component proximal end are arranged together and the outer component proximal end and the middle component proximal end are arranged together and the outer component distal end and the middle component distal end are arranged together; and
an interior component having an interior proximal end and an interior distal end, the interior component configured to be arranged within the middle component such that the outer component proximal end, the middle component proximal end, and the interior component proximal end are arranged together and the outer component distal end, the middle component distal end, and the interior component distal end are arranged together.

14. The method of claim 13, further comprising:
providing an expansion and retraction mechanism removably coupled to the interior component to facilitate expanding a volume of the interior component.

15. The method of claim 14, further comprising:
facilitating retraction and removal of the expansion and retraction mechanism and the surgical packing material after a post-operative period.

16. The method of claim 13, further comprising:
configuring the middle component for:
insertion of a douche device into the middle component in situ,
coupling of the douche device to the tube portion, and
expressing of douche fluid from the douche device, through the tube portion, the proximal end aperture, and into the plurality of channels.

17. The method of claim 16, further comprising configuring the proximal end aperture and the plurality of channels such that douche fluid is provided to an external surface of the outer component.

18. The method of claim 13, further comprising providing a suturing tab portion coupled with at least the interior component to facilitate selective securing of the flushing stent device to the patient by suturing the flushing stent device to the patient.

19. The method of claim 18, further comprising configuring the suturing tab portion for selective removable coupling of a catheter to a notched end of the suturing tab portion.

20. The method of claim 18, wherein the suturing portion further comprises a plurality of suturing tab apertures arranged proximate an edge of the suturing tab portion to facilitate the selective securing of the flushing stent device to a patient by applying sutures to the suturing tab portion.

21. The method of claim 13, further comprising configuring the tube position to extend past the distal end such that the tube portion is accessible via a vaginal canal of the patient when the flushing stent device is in situ above a pelvic floor musculature of the patient.

22. The method of claim 21, further comprising extending the tube portion.
